(19) United States
(12) Patent Application Publication
(10) Pub. No.: US 2026/0367025 A1
(43) Pub. Date: Nov. 19, 2026

(54) HYDROGEN DETECTION DEVICE AND HYDROGEN DETECTION METHOD

(71) Applicant: Nuvoton Technology Corporation Japan, Kyoto (JP)

(72) Inventors: Kazunari HOMMA, Kyoto (JP); Chuanyang HUANG, Kyoto (JP); Koji KATAYAMA, Kyoto (JP); Tomohiro MINAGAWA, Kyoto (JP); Ken KAWAI, Kyoto (JP); Satoru ITO, Kyoto (JP)

(21) Appl. No.: 19/725,959

(22) Filed: Jun. 30, 2026

Related U.S. Application Data
(63) Continuation of application No. PCT/JP2025/001843, filed on Jan. 22, 2025.

(30) Foreign Application Priority Data
Jan. 25, 2024 (JP) 2024-009494

(51) Int. Cl. G01N 27/12; G01N 33/00

(52) U.S. Cl. CPC G01N 27/122; G01N 33/005

(57) ABSTRACT

A hydrogen detection device includes a hydrogen sensor element, a first reference element, and a second reference element that are provided on a semiconductor substrate and connected in series. Each of the hydrogen sensor element, the first reference element, and the second reference element includes a layered body of the same structure. The hydrogen sensor element includes an opening that is not covered by an insulating film, etc. and through which a surface of a second electrode opposite to a principal surface of the second electrode is exposed to an external environment, each of the first reference element and the second reference element includes no opening through which a surface of a second electrode opposite to a principal surface of the second electrode is exposed to the outside, and the first reference element and the second reference element have mutually different lengths in a direction in which a current flows.

### CROSS REFERENCE TO RELATED APPLICATION

This is a continuation application of PCT International Patent Application No. PCT/JP 2025/001843 filed on Jan. 22, 2025, designating the United States of America, which is based on and claims priority of Japanese Patent Application No. 2024-009494 filed on Jan. 25, 2024. The entire disclosures of the above-identified applications, including the specifications, drawings and claims are incorporated herein by reference in their entirety.

### FIELD

The present disclosure relates to a hydrogen detection device and a hydrogen detection method, and in particular relates to a hydrogen detection device that includes a sensor chip including a resistive element provided on a semiconductor substrate.

### BACKGROUND

A hydrogen detection device that includes a sensor chip including a resistive element provided on a semiconductor substrate has been conventionally proposed (see Patent Literature (PTL) 1, for example).

### CITATION LIST

### Patent Literature

PTL 1: Japanese Unexamined Patent Application Publication No. 2019-152451

### SUMMARY

### Technical Problem

However, because a bridge circuit including four resistive elements is included in the sensor chip disclosed in PTL 1, the area of the sensor chip provided on the semiconductor substrate is large.

In view of the above, the present disclosure provides: a hydrogen detection device that includes a sensor chip that is realized with a smaller area than that of a conventional one and can detect hydrogen with high sensitivity; and a hydrogen detection method.

### Solution to Problem

A hydrogen detection device according to an aspect of the present disclosure is a hydrogen detection device that detects hydrogen and includes a first resistive element, a second resistive element, and a third resistive element that are provided on a semiconductor substrate and electrically connected in series. Each of the first resistive element, the second resistive element, and the third resistive element includes: a first electrode including a principal surface and a second electrode including a principal surface, the principal surface of the first electrode and the principal surface of the second electrode facing each other; a metal oxide layer disposed in contact with the principal surface of the first electrode and the principal surface of the second electrode; and an insulating film covering the first electrode, the second electrode, and the metal oxide layer, the first resistive element includes an opening that is not covered by the insulating film and through which an other surface of the second electrode opposite to the principal surface is exposed to an external environment, each of the second resistive element and the third resistive element includes no opening through which an other surface of the second electrode opposite to the principal surface is exposed to the external environment, and the second resistive element and the third resistive element have different lengths in a direction in which a current flows.

A hydrogen detection method according to an aspect of the present disclosure is a method for detecting hydrogen and includes: preparing the hydrogen detection device; applying a current to the first resistive element, the second resistive element, and the third resistive element that are included in the hydrogen detection device prepared and are connected in series; measuring a voltage across both ends of each of the first resistive element, the second resistive element, and the third resistive element when the current is applied in the applying; and generating a signal indicating hydrogen concentration by using the voltage across both ends of each of the first resistive element, the second resistive element, and the third resistive element obtained in the measuring, and outputting the signal.

### Advantageous Effects

The present disclosure provides: a hydrogen detection device that includes a sensor chip that is realized with a smaller area than that of a conventional one and can detect hydrogen with high sensitivity; and a hydrogen detection method. In particular, by configuring the reference resistive elements such that hydrogen does not reach either the second electrode or the metal oxide layer thereof, the influence of hydrogen on the reference resistive elements is reliably eliminated.

### BRIEF DESCRIPTION OF DRAWINGS

These and other advantages and features will become apparent from the following description thereof taken in conjunction with the accompanying Drawings, by way of non-limiting examples of embodiments disclosed herein.

FIG. 1

FIG. 1 is a block diagram illustrating a configuration of a hydrogen detection device according to an embodiment.

FIG. 2A

FIG. 2A is a cross-sectional view illustrating an example of a configuration of a sensor chip illustrated in FIG. 1.

FIG. 2B

FIG. 2B is a top view illustrating the example of the configuration of the sensor chip illustrated in FIG. 2A.

FIG. 3

FIG. 3 is a flowchart illustrating a method for manufacturing the sensor chip illustrated in FIG. 2A and FIG. 2B.

FIG. 4

FIG. 4 is a flowchart illustrating a procedure for detecting hydrogen (i.e., a hydrogen detection method) performed by the hydrogen detection device according to the embodiment.

FIG. 5

FIG. 5 is a diagram illustrating an example of a plot showing the relationship between the length of the internal resistance of each of a first reference element and a second reference element (the horizontal axis) and a voltage across both ends of each of the first reference element and the second reference element when a current is applied (the vertical axis).

FIG. 6

FIG. 6 is a diagram for describing a result of a hydrogen concentration detection experiment performed by the hydrogen detection device according to the embodiment.

FIG. 7A

FIG. 7A is a cross-sectional view illustrating an example of a configuration of a sensor chip according to a variation of the embodiment.

FIG. 7B

FIG. 7B is a top view illustrating the example of the configuration of the sensor chip illustrated in FIG. 7A.

FIG. 8

FIG. 8 is a flowchart illustrating a method for manufacturing the sensor chip illustrated in FIG. 7A and FIG. 7B.

### DESCRIPTION OF EMBODIMENT

Hereinafter, an embodiment of the present disclosure will be described in detail with reference to the Drawings. It should be noted that the embodiment described below shows a specific example of the present disclosure. The numerical values, shapes, materials, constituent elements, the arrangement and connection of the constituent elements, steps, the order of the steps, etc., in the following embodiment are mere examples, and therefore do not intend to limit the present disclosure. Moreover, each drawing is not necessarily an exact depiction. In each drawing, elements that have substantially the same configuration share the same reference signs, and overlapping description thereof is omitted or simplified. Moreover, “connection” means electrical connection, and includes not only a case where two circuit elements are directly connected to each other but also a case where two circuit elements are indirectly connected to each other in a state where another circuit element is interposed between the two circuit elements.

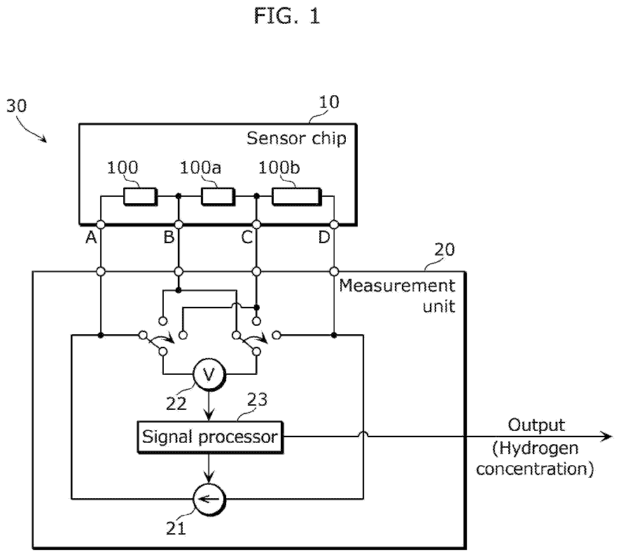

FIG. 1 is a block diagram illustrating a configuration of hydrogen detection device 30 according to the embodiment. Hydrogen detection device 30 includes sensor chip 10 and measurement unit 20.

Sensor chip 10 includes hydrogen sensor element 100 that is an example of a first resistive element, first reference element 100a that is an example of a second resistive element, and second reference element 100b that is an example of a third resistive element that are connected in series and provided on a semiconductor substrate. Moreover, sensor chip 10 includes, as terminals for connection to the outside, terminal A that is connected to one end of hydrogen sensor element 100, terminal B that is connected to a connection point between the other end of hydrogen sensor element 100 and one end of first reference element 100a, terminal C that is connected to a connection point between the other end of first reference element 100a and one end of second reference element 100b, and terminal D that is connected to the other end of second reference element 100b.

Here, hydrogen sensor element 100 is a variable resistive element that has a structure to be exposed to hydrogen and has a resistance value that varies depending on hydrogen concentration. Meanwhile, first reference element 100a and second reference element 100b are fixed resistive elements for reference that have different lengths in a direction in which a current flows and that do not respond to hydrogen. More specifically, first reference element 100a and second reference element 100b are provided to cancel the temperature dependence of the resistance value of hydrogen sensor element 100 and to remove an unnecessary voltage generated due to an unnecessary resistance component (i.e., an external resistance that is a parasitic resistance that does not contribute to reaction with hydrogen) included in hydrogen sensor element 100, and each have a structure that is basically the same as that of hydrogen sensor element 100 but is not exposed to hydrogen.

In the present disclosure, the second resistive element and the third resistive element are each configured such that hydrogen does not reach either the second electrode or the metal oxide layer thereof.

Such a configuration is realized, for example, by not providing an opening through which the second electrode is exposed, or by providing an opening whose inner surfaces are covered by a hydrogen-impermeable film.

It should be noted that hydrogen sensor element 100, first reference element 100a, and second reference element 100b need only be connected in series, the connection order thereof is not limited to that illustrated in the diagram, and first reference element 100a, hydrogen sensor element 100, and second reference element 100b may be connected in the stated order, for example.

Measurement unit 20 is a circuit that detects, using sensor chip 10, hydrogen concentration in an environment in which sensor chip 10 is located and that outputs a signal indicating the hydrogen concentration detected, and includes current source 21, voltmeter 22, and signal processor 23. Current source 21 is a circuit that applies a predetermined constant current between terminal A and terminal D of sensor chip 10 in accordance with an instruction from signal processor 23, and includes, for example, a transistor or the like. Voltmeter 22 is a circuit that measures, in accordance with an instruction from signal processor 23: a voltage across both ends of hydrogen sensor element 100 (i.e., voltage drop between terminals A and B); a voltage across both ends of first reference element 100a (i.e., voltage drop between terminals B and C); and a voltage across both ends of second reference element 100b (i.e., voltage drop between terminals C and D), while switching between the three elements, and voltmeter 22 includes, for example, a switch circuit, a voltage amplifier, an A/D converter, or the like. Signal processor 23 is a circuit that: instructs current source 21 to perform current application; reads out the three types of measured voltages from voltmeter 22; generates a signal indicating hydrogen concentration based on the three types of measured voltages read out; and outputs the signal, and includes, for example, a microprocessor or the like that executes a built-in program.

It should be noted that measurement unit 20 may be a circuit provided on the same semiconductor substrate as sensor chip 10, or may be a circuit, a measuring device, a computer device for measurement, or the like that is provided in a different substrate or device that is connected to sensor chip 10. Moreover, voltmeter 22 is not limited to being configured to measure the three types of voltages while switching between the three elements, and may be configured as a group of three voltmeters that measure the three types of voltages in parallel.

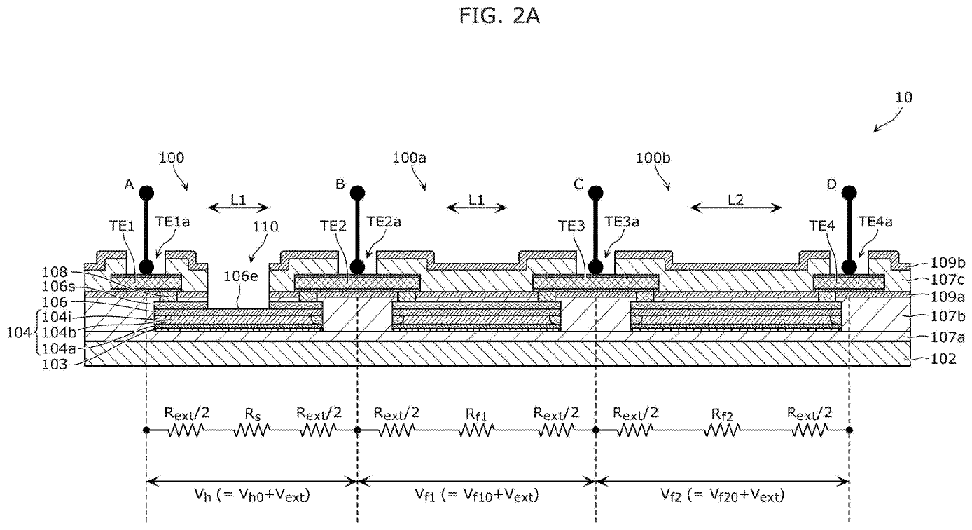

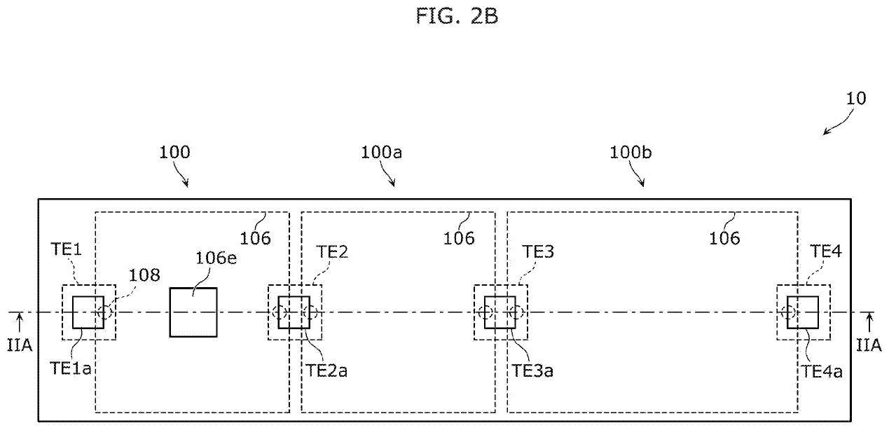

FIG. 2A is a cross-sectional view illustrating an example of a configuration of sensor chip 10 illustrated in FIG. 1. Here, for explanation, in addition to a cross-sectional view of hydrogen sensor element 100, first reference element 100a, and second reference element 100b that are connected in series, schematic wiring lines each representing a connection to a different one of terminals A to D are illustrated in an upper portion of the cross-sectional view and an equivalent circuit (resistances Rext, Rs, Rf1, Rf2, and the like) of sensor chip 10 and voltages (Vh, Vf1, Vf2, and the like) generated by the resistances when a current is applied are illustrated in a lower portion of the cross-sectional view. FIG. 2B is a top view illustrating the example of the configuration of sensor chip 10 illustrated in FIG. 2A. It should be noted that FIG. 2A schematically illustrates the cross section taken along line IIA-IIA and viewed in the arrow direction in FIG. 2B.

Hydrogen sensor element 100 located at the left end of sensor chip 10 illustrated in FIG. 2A is provided above semiconductor substrate 102 and insulating film 107a thereon, and includes, as main constituent elements: first electrode 103 including a principal surface and second electrode 106 including a principal surface, the principal surface of first electrode 103 and the principal surface of second electrode 106 facing each other; metal oxide layer 104 disposed in contact with the principal surface (i.e., the upper surface) of first electrode 103 and the principal surface (i.e., the lower surface) of second electrode 106; and insulating films (insulating films 107a to 107c, 109a, and 109b) covering first electrode 103, second electrode 106, and metal oxide layer 104. For exposing hydrogen sensor element 100 to hydrogen, insulating films 107b, 107c, 109a, and 109b include, at a central position of an other surface (i.e., the upper surface) of second electrode 106 that is opposite to the principal surface, opening 110 that is not covered by the insulating films so as to be exposed to the external environment. In the present embodiment, metal layer 106s is also removed to expose second electrode 106 through opening 110.

Hydrogen sensor element 100 described above includes: first terminal TE1 for connection to terminal A; and second terminal TE2 for connection to both terminal B and first reference element 100a. It should be noted that second terminal TE2 is shared by hydrogen sensor element 100 and first reference element 100a. Each of first terminal TE1 and second terminal TE2 is connected, through via 108, to the other surface of second electrode 106. Hydrogen sensor element 100 changes its resistance value depending on the hydrogen concentration at opening 110 when a current is applied in the horizontal direction in FIG. 2A.

First reference element 100a located at the center of sensor chip 10 illustrated in FIG. 2A and second reference element 100b located at the right end of sensor chip 10 illustrated in FIG. 2A each have basically the same structure as that of hydrogen sensor element 100. In other words, each of first reference element 100a and second reference element 100b is provided above semiconductor substrate 102 and insulating film 107a thereon, and includes, as main constituent elements: first electrode 103 including a principal surface and second electrode 106 including a principal surface, the principal surface of first electrode 103 and the principal surface of second electrode 106 facing each other; metal oxide layer 104 disposed in contact with the principal surface (i.e., the upper surface) of first electrode 103 and the principal surface (i.e., the lower surface) of second electrode 106; and insulating films (insulating films 107a to 107c, 109a, and 109b) covering first electrode 103, second electrode 106, and metal oxide layer 104.

By contrast, hydrogen does not reach either second electrode 106 or metal oxide layer 104 of first reference element 100a and second reference element 100b. As a result, no reaction with hydrogen-containing gas occurs in first reference element 100a and second reference element 100b. As described above, first reference element 100a and second reference element 100b are fixed resistive elements whose resistance values do not depend on hydrogen concentration. These reference elements are provided to cancel temperature dependence of the resistance value of hydrogen sensor element 100 and to remove an external resistance that is an unnecessary parasitic resistance included in hydrogen sensor element 100.

First reference element 100a includes: second terminal TE2 for connection to both terminal B and hydrogen sensor element 100; and third terminal TE3 for connection to both terminal C and second reference element 100b. It should be noted that third terminal TE3 is shared by first reference element 100a and second reference element 100b. Moreover, second reference element 100b includes: third terminal TE3 for connection to both terminal C and first reference element 100a; and fourth terminal TE4 for connection to terminal D.

Here, the ratio of the length of hydrogen sensor element 100, the length of first reference element 100a, and the length of second reference element 100b in a direction in which a current flows (the horizontal direction in FIG. 2A) is 1:1:2 in the present embodiment. Moreover, as illustrated in the upper portion of the cross-sectional view of FIG. 2A, hydrogen sensor element 100 includes opening 110 having length L1 in the direction in which a current flows, first reference element 100a has a structure equivalent to a structure in which opening 110 having length L1 in hydrogen sensor element 100 is closed by an insulating film or the like, and second reference element 100b has a structure equivalent to a structure in which opening 110 in hydrogen sensor element 100 has length L2 (=2×L1) and is closed by an insulating film or the like.

It should be noted that the ratio of the length of hydrogen sensor element 100, the length of first reference element 100a, and the length of second reference element 100b is not limited to 1:1:2. In order to maintain the accuracy of calculation (calculation of a straight line, calculation of a voltage ratio, etc.) in signal processor 23 described later, the length of first reference element 100a is, for example, a length similar to that of hydrogen sensor element 100 but is not necessarily limited thereto, and may be 0.5 to 2 times as long as that of hydrogen sensor element 100. Moreover, the length of second reference element 100b is, for example, at least 2 times as long as that of first reference element 100a but is not necessarily limited thereto, and may be at least 0.5 times as long as that of first reference element 100a.

The details of each constituent element shared among hydrogen sensor element 100, first reference element 100a, and second reference element 100b included in sensor chip 10 will be described below.

First electrode 103 is a planar electrode and includes two surfaces. Of the two surfaces of first electrode 103, one surface (i.e., the upper surface in FIG. 2A) is in contact with metal oxide layer 104, and the other surface (i.e., the lower surface in FIG. 2A) is in contact with insulating film 107a. In FIG. 2B, first electrode 103 is in a rectangular shape of the same size as second electrode 106. First electrode 103 may be made of, for example, a material such as tungsten, nickel, tantalum, titanium, aluminum, tantalum nitride, or titanium nitride, which has a standard electrode potential lower than that of metals included in metal oxides. The higher the standard electrode potential is, the more resistant the material is to oxidation. First electrode 103 in FIG. 2A is formed of, for example, transition metal nitride such as tantalum nitride (TaN) or titanium nitride (TiN), or a layered structure thereof.

Metal oxide layer 104 is disposed between the principal surface of first electrode 103 and the principal surface of second electrode 106 facing each other, includes a metal oxide serving as a gas-sensitive resistance film, and has a resistance value that reversibly changes according to the presence or absence of hydrogen-containing gas in gas that is in contact with second electrode 106. It is sufficient so long as metal oxide layer 104 has a property that enables its resistance to change according to hydrogen. The base metal of metal oxide layer 104 may be at least one selected from aluminum (Al) and transition metals such as tantalum (Ta), hafnium (Hf), titanium (Ti), zirconium (Zr), niobium (Nb), tungsten (W), nickel (Ni), and iron (Fe).

Metal oxide layer 104 illustrated in FIG. 2A includes: first layer 104a that is in contact with first electrode 103; second layer 104b that is in contact with first layer 104a and second electrode 106; and insulating isolation layer 104i. The degree of oxygen deficiency of second layer 104b is lower than that of first layer 104a. For example, first layer 104a is TaOₓ. Second layer 104b is Ta₂O₅ whose degree of oxygen deficiency is lower than that of first layer 104a. Moreover, metal oxide layer 104 includes insulating isolation layer 104i at the perimeter of first electrode 103 in plan view.

Here, the plan view is the plan view of semiconductor substrate 102, and means viewing sensor chip 10 according to the present disclosure from a viewpoint in the layer-stacking direction in FIG. 2A, in other words, viewing from a viewpoint in the direction normal to any of the surfaces of first electrode 103 and second electrode 106 that are planar, for example. For example, the plan view refers to viewing the top surface of sensor chip 10 illustrated in FIG. 2B.

In hydrogen sensor element 100, the resistance value of metal oxide layer 104 described above decreases depending on the hydrogen-containing gas that is in contact with second electrode 106 through opening 110 (i.e., the resistance value of metal oxide layer 104 decreases as the amount of the hydrogen-containing gas increases). Specifically, when hydrogen-containing gas is present in detection-target gas, hydrogen atoms are dissociated from the hydrogen-containing gas at second electrode 106. The hydrogen atoms dissociated enter metal oxide layer 104 and form an impurity level. Particularly, the hydrogen atoms dissociated concentrate in the vicinity of the interface with second electrode 106 and decrease the apparent thickness of second layer 104b. As a result, the resistance value of metal oxide layer 104 decreases. By contrast, in first reference element 100a and second reference element 100b, hydrogen does not reach either second electrode 106 or metal oxide layer 104. Accordingly, the dissociation of hydrogen atoms and the subsequent change in resistance do not occur in these reference elements.

Second electrode 106 is a planar electrode that is capable of dissociating hydrogen and includes two surfaces. Of the two surfaces of second electrode 106, one surface (i.e., the lower surface in FIG. 2A) is in contact with metal oxide layer 104, and the other surface (i.e., the upper surface in FIG. 2A) is in contact with metal layer 106s (in hydrogen sensor element 100, also with the outside air). In hydrogen sensor element 100, second electrode 106 includes, in opening 110, exposed portion 106e that is exposed to the outside air. Second electrode 106 is made of, for example, a material having a function of catalyzing dissociation of hydrogen atoms from gas molecules containing hydrogen atoms, such as: a noble metal such as platinum (Pt), iridium (Ir), or palladium (Pd); nickel (Ni); or an alloy containing at least one of these. It is assumed that second electrode 106 in FIG. 2A is platinum (Pt). Four terminals, that are, first terminal TE1, second terminal TE2, third terminal TE3, and fourth terminal TE4 are connected to second electrode 106 through via 108. First terminal TE1, second terminal TE2, third terminal TE3, and fourth terminal TE4 are connected to terminals A, B, C, and D of sensor chip 10 through openings TE1a, TE2a, TE3a, and TE4a, respectively, by wiring.

As illustrated in FIG. 2B, first terminal TE1 and second terminal TE2 are arranged at positions between which exposed portion 106e is provided in the plan view of second electrode 106. With this arrangement, application of a current between first terminal TE1 and second terminal TE2 causes passage of the current through exposed portion 106e of second electrode 106, that is, causes the current to flow through exposed portion 106e. The passage of the current through exposed portion 106e of second electrode 106 is considered to activate the hydrogen dissociation function of exposed portion 106e. In contrast, in first reference element 100a and second reference element 100b, hydrogen does not reach either second electrode 106 or metal oxide layer 104. Therefore, the catalytic dissociation of hydrogen and the activation of hydrogen-sensitive resistance do not occur in these reference elements.

It should be noted that insulating films 107a to 107c and insulating films 109a and 109b that cover main components of hydrogen sensor element 100 are each formed of a silicon oxide film, a silicon nitride film, or the like.

Moreover, metal layer 106s is provided on the upper surface of second electrode 106 excluding opening 110. Metal layer 106s is made of, for example, TiAlN and is formed as an etching stopper for forming a via; however, metal layer 106s is not necessarily provided.

It should be noted that although an example in which metal oxide layer 104 has a two-layer configuration including first layer 104a made of TaOₓ and second layer 104b made of Ta₂O₅ whose degree of oxygen deficiency is low has been illustrated in FIG. 2A, metal oxide layer 104 may have a single layer configuration including a layer made of TaOₓ or Ta₂O₅ whose degree of oxygen deficiency is low.

Equivalent resistances (Rext, Rs, Rf1, and Rf2) in sensor chip 10 and voltages (Vh, Vf1, Vf2, and the like) generated by the resistances when a current is applied are illustrated in the lower portion of the cross-sectional view in FIG. 2A.

As illustrated in FIG. 2A, the resistance of hydrogen sensor element 100 is composed of a series connection of: resistance Rs that is a resistance component at a portion of second electrode 106 directly below opening 110 having length L1 (i.e., exposed portion 106e of second electrode 106); an unnecessary parasitic resistance from resistance Rs to terminal A (i.e., half of external resistance Rext (Rext/2)); and an unnecessary parasitic resistance from resistance Rs to terminal B (i.e., half of external resistance Rext (Rext/2)). External resistance Rext is equivalent to the total value of parasitic resistances that do not contribute to reaction with hydrogen in hydrogen sensor element 100.

As illustrated in FIG. 2A, the resistance of first reference element 100a is composed of a series connection of: resistance Rf1 that is a resistance component at a portion having length L1 corresponding to resistance component Rs of hydrogen sensor element 100 (hereinafter, also referred to as “internal resistance Rf1”); an unnecessary parasitic resistance from internal resistance Rf1 to terminal B (i.e., half of external resistance Rext (Rext/2)); and an unnecessary parasitic resistance from internal resistance Rf1 to terminal C (i.e., half of external resistance Rext (Rext/2)).

As illustrated in FIG. 2A, the resistance of second reference element 100b is composed of a series connection of: resistance Rf2 that is a resistance component at a portion having length L2 corresponding to resistance component Rs of hydrogen sensor element 100 (hereinafter, also referred to as “internal resistance Rf2”); an unnecessary parasitic resistance from internal resistance Rf2 to terminal C (i.e., half of external resistance Rext (Rext/2)); and an unnecessary parasitic resistance from internal resistance Rf2 to terminal D (i.e., half of external resistance Rext (Rext/2)).

It should be noted that a structure from the terminal A-side end of the portion of second electrode 106 directly below opening 110 having length L1 (i.e., exposed portion 106e of second electrode 106) to terminal A in hydrogen sensor element 100, a structure from the terminal B-side end of exposed portion 106e to terminal B, a structure from the terminal B-side end of internal resistance Rf1 having length L1 to terminal B in first reference element 100a, a structure from the terminal C-side end of internal resistance Rf1 having length L1 to terminal C, a structure from the terminal C-side end of internal resistance Rf2 having length L2 to terminal C in second reference element 100b, and a structure from the terminal D-side end of internal resistance Rf2 having length L2 to terminal D are the same, and each of the structures has a resistance value that is half of external resistance Rext (Rext/2).

In view of the above, as illustrated in the lower portion of FIG. 2A, when a current is applied, voltage Vh generated in hydrogen sensor element 100 is represented as the sum of voltage Vh0 generated by resistance Rs that changes depending on hydrogen concentration and voltage Vext generated by unnecessary external resistance Rext (=Rext/2+Rext/2) that is not dependent on hydrogen concentration. Moreover, voltage Vf1 generated in first reference element 100a is represented as the sum of voltage Vf10 generated by fixed internal resistance Rf1 and voltage Vext generated by unnecessary external resistance Rext (=Rext/2+Rext/2) that is not dependent on hydrogen concentration. Furthermore, voltage Vf2 generated in second reference element 100b is represented as the sum of voltage Vf20 generated by fixed internal resistance Rf2 and voltage Vext generated by unnecessary external resistance Rext (=Rext/2+Rext/2) that is not dependent on hydrogen concentration.

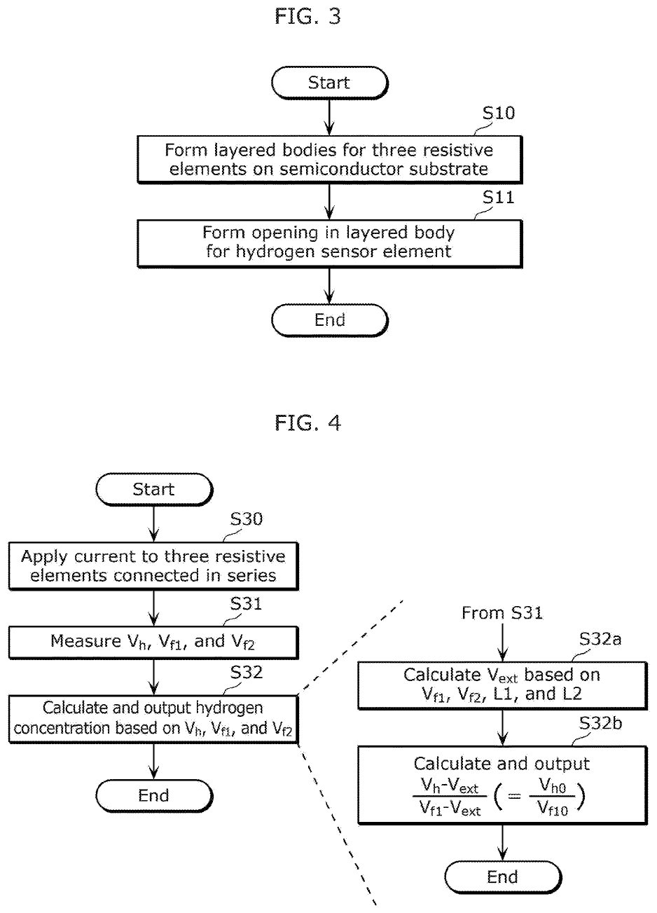

FIG. 3 is a flowchart illustrating a method for manufacturing sensor chip 10 illustrated in FIG. 2A and FIG. 2B. First, film formation and photolithography (pattern transferring and etching) for forming layered bodies for three resistive elements (hydrogen sensor element 100, first reference element 100a, and second reference element 100b) are repeatedly performed on semiconductor substrate 102 to form layered bodies each including, from the bottom: semiconductor substrate 102; insulating film 107a as an inter-layer insulating film containing, for example, plasma tetra ethoxy silane (P-TEOS); first electrode 103 containing, for example, TaN or TiN; metal oxide layer 104 containing, for example, a layered body of Ta₂O₅ and TaO_{1.5}; second electrode 106 containing, for example, Pt; metal layer 106s containing, for example, TiAlN; insulating film 107b as an inter-layer insulating film containing, for example, P-TEOS; insulating film 109a as a protective film containing, for example, plasma silicon oxynitride film (P-SiON); first terminal TE1 to fourth terminal TE4 as electrodes each containing, for example, Au; insulating film 107c as an inter-layer insulating film containing, for example, high density plasma nitrogen doped glass (HDP-NSG); and insulating film 109b as a protective film containing, for example, P-SiON (layered body forming step S10). By performing this step, an intermediate product of hydrogen sensor element 100 in which opening 110 has not yet been formed, and finished products of first reference element 100a and second reference element 100b are manufactured.

Next, photolithography (pattern transferring and etching) is performed on the layered body (i.e., the intermediate product of hydrogen sensor element 100) to remove, in a rectangular shape, part of metal layer 106s, insulating film 107b, insulating film 109a, insulating film 107c, and insulating film 109b so as to expose at least part of the upper surface of second electrode 106, and thus opening 110 of hydrogen sensor element 100 is formed (opening forming step S11). By performing this step, hydrogen sensor element 100 is finished and the manufacture of sensor chip 10 is completed.

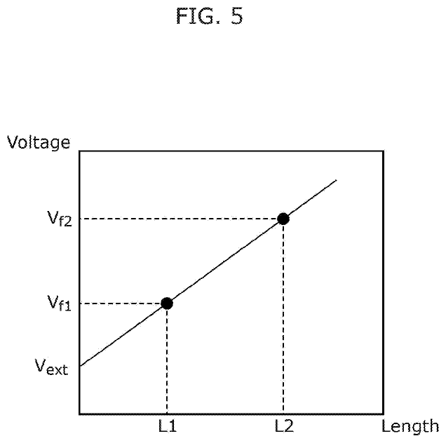

Next, an operation of hydrogen detection device 30 configured as described above will be described. FIG. 4 is a flowchart illustrating a procedure for detecting hydrogen (i.e., a hydrogen detection method) performed by hydrogen detection device 30 according to the embodiment.

First, after preparation, namely, installation of hydrogen detection device 30 in a target environment, has been completed, hydrogen detection device 30 applies a current to hydrogen sensor element 100, first reference element 100a, and second reference element 100b that are connected in series (current applying step S30). Namely, in measurement unit 20 of hydrogen detection device 30, current source 21 applies a predetermined constant current between terminal A and terminal D of sensor chip 10, in accordance with an instruction from signal processor 23.

Next, hydrogen detection device 30 measures a voltage across both ends of each of hydrogen sensor element 100, first reference element 100a, and second reference element 100b when a current is applied in current applying step S30 (voltage measuring step S31). Namely, in measurement unit 20 of hydrogen detection device 30, voltmeter 22 measures, in accordance with an instruction from signal processor 23: voltage Vh across both ends of hydrogen sensor element 100; voltage Vf1 across both ends of first reference element 100a; and voltage Vf2 across both ends of second reference element 100b, which are voltages when a current is applied, while switching between the three elements.

Next, hydrogen detection device 30 generates a signal indicating hydrogen concentration, using voltage Vh across both ends of hydrogen sensor element 100, voltage Vf1 across both ends of first reference element 100a, and voltage Vf2 across both ends of second reference element 100b obtained in voltage measuring step S31, and outputs the signal (signal processing step S32).

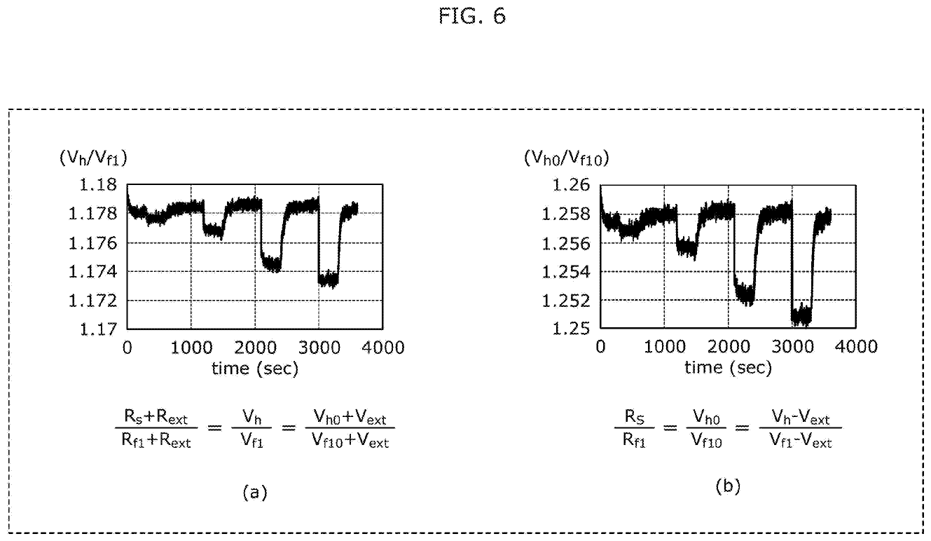

Specifically, in measurement unit 20 of hydrogen detection device 30, based on voltage Vf1 across both ends of first reference element 100a and voltage Vf2 across both ends of second reference element 100b obtained in voltage measuring step S31, known length L1 of a portion corresponding to internal resistance Rf1 of first reference element 100a, and known length L2 of a portion corresponding to internal resistance Rf2 of second reference element 100b, signal processor 23 first draws a straight line passing through a point (L1, Vf1) and a point (L2, Vf2) in a two-dimensional coordinate system illustrated in FIG. 5 and calculates, as voltage Vext generated by external resistance Rext, the intercept with the vertical axis (S32a). FIG. 5 is a diagram illustrating an example of a plot showing the relationship between the length of each of internal resistances Rf1 and Rf2 of first reference element 100a and second reference element 100b (the horizontal axis) and a voltage across both ends of each of first reference element 100a and second reference element 100b when a current is applied (the vertical axis). Voltage Vext generated by external resistance Rext is calculated based on the straight line passing through the two points, by utilizing the fact that voltage Vf1 across both ends of first reference element 100a and voltage Vf2 across both ends of second reference element 100b are each represented by the sum of: a voltage component generated in proportion to the length of a corresponding one of internal resistances Rf1 and Rf2; and offset voltage Vext generated by fixed external resistance Rext.

Then, signal processor 23: generates, as a signal indicating hydrogen concentration, a voltage ratio that is represented by Expression 1 shown below where Vext denotes the voltage calculated, Vh denotes the voltage across both ends of hydrogen sensor element 100 obtained in voltage measuring step S31 described above, and Vf1 denotes the voltage across both ends of first reference element 100a; and outputs the signal to the outside (S32b).

(Vh−Vext)/(Vf1−Vext) (expression 1)

It should be noted that Expression 1 shown above represents Expression 2 shown below since Vh−Vext=Vh0 holds in the numerator of Expression 1 shown above and Vf1−Vext=Vf0 holds in the denominator of Expression 1 shown above.

Vh0/Vf10 (Expression 2)

As can be seen from Expression 2 shown above, numerator Vh0 in Expression 2 shown above represents only a voltage generated by resistance Rs that changes depending on hydrogen concentration, while excluding a voltage generated by unnecessary external resistance Rext in hydrogen sensor element 100. Moreover, denominator Vf10 in Expression 2 shown above also represents only a voltage generated by fixed internal resistance Rf1 as a reference, while excluding a voltage generated by unnecessary external resistance Rext.

Then, the voltage ratio represented by Expression 2 shown above is also a ratio of net resistance Rs of hydrogen sensor element 100 that is sensitive to hydrogen to net internal resistance Rf1 of first reference element 100a that is not sensitive to hydrogen (Rs/Rf1), in which hydrogen sensor element 100 and first reference element 100a have been provided on the same semiconductor substrate and have the same structure. Accordingly, it can be said that the voltage ratio represented by Expression 2 shown above is a value that indicates hydrogen concentration with high sensitivity, while suppressing temperature dependence and removing the component of unnecessary external resistance Rext of hydrogen sensor element 100.

FIG. 6 is a diagram for describing a result of a hydrogen concentration detection experiment performed by hydrogen detection device 30 according to the embodiment. This result of the experiment shows temporal changes of two types of voltage ratios (i.e., reaction amounts) obtained from sensor chip 10 when the atmosphere in which sensor chip 10 is placed is sequentially switched in four stages from a low hydrogen concentration state to a high hydrogen concentration state. (a) of FIG. 6 shows a temporal change of a voltage ratio (Vh/Vf1) according to a reference example in a state where voltage Vext due to external resistance Rext of each of hydrogen sensor element 100 and first reference element 100a is included, meanwhile, (b) of FIG. 6 shows a temporal change of a voltage ratio (Vh0/Vf10) according to the embodiment in a state where voltage Vext due to external resistance Rext of each of hydrogen sensor element 100 and first reference element 100a is excluded.

It should be noted that the voltage ratio (Vh/Vf1) according to the reference example shown in (a) of FIG. 6 can also be expressed as (Vh0+Vext)/(Vf10+Vext) as well as (Rs+Rext)/(Rf1+Rext), as illustrated in the drawing. Meanwhile, the voltage ratio (Vh0/Vf10) according to the embodiment shown in (b) of FIG. 6 can also be expressed as (Vh−Vext)/(Vf1−Vext) as well as (Rs/Rf1), as illustrated in the drawing.

In (b) of FIG. 6 according to the embodiment, the voltage ratio (i.e., reaction amount) is about 1.5 times as great as that of (a) of FIG. 6 according to the reference example. In other words, the sensitivity of detecting the reaction amount in the embodiment in which the voltage due to external resistance Rext is excluded is increased to about 1.5 times as compared with that in the reference example in which the voltage due to external resistance Rext is included. Accordingly, it can be seen that hydrogen detection device 30 according to the embodiment can detect hydrogen at low concentrations with high sensitivity as compared with the hydrogen detection device according to the reference example.

It should be noted that sensor chip 10 included in hydrogen detection device 30 according to the embodiment is not limited to a sensor chip that has the structure illustrated in FIG. 2A and FIG. 2B, and may be a sensor chip according to a variation or the like described below.

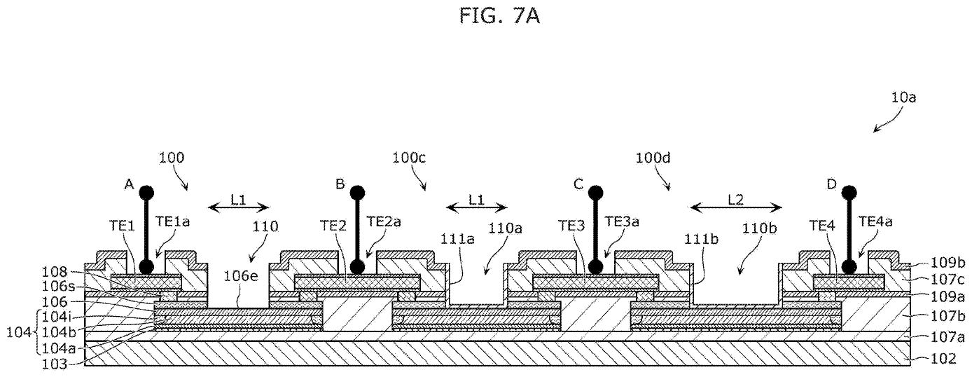

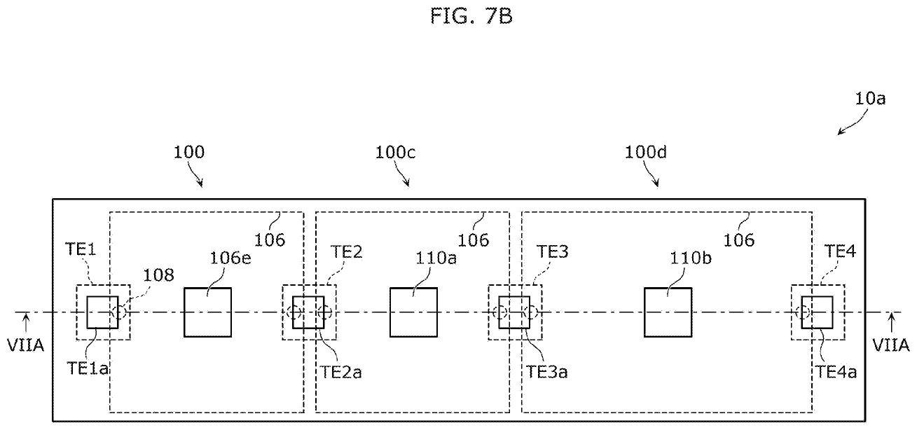

FIG. 7A is a cross-sectional view illustrating an example of a configuration of sensor chip 10a according to a variation of the embodiment. FIG. 7B is a top view illustrating the example of the configuration of sensor chip 10a illustrated in FIG. 7A. It should be noted that FIG. 7A schematically illustrates the cross section taken along line VIIA-VIIA and viewed in the arrow direction in FIG. 7B.

In the present variation, sensor chip 10a includes hydrogen sensor element 100 that is an example of a first resistive element, first reference element 100c that is an example of a second resistive element, and second reference element 100d that is an example of a third resistive element that are connected in series and provided on a semiconductor substrate.

Sensor chip 10a according to the present variation is different from sensor chip 10 according to the embodiment illustrated in FIG. 2A and FIG. 2B in that, in sensor chip 10a according to the present variation, layered bodies for hydrogen sensor element 100, first reference element 100c, and second reference element 100d are formed in the same manufacturing process and then openings 110, 110a, and 110b are formed, and thereafter the inner side surfaces and the bottom surfaces of opening 110a of first reference element 100c and opening 110b of second reference element 100d are covered by hydrogen-impermeable films 111a and 111b. It should be noted that each of hydrogen-impermeable films 111a and 111b is a film that does not allow hydrogen to pass through, and is, for example, a film made of a silicon oxide film, a silicon nitride film, a silicon oxynitride film, or the like. Accordingly, even though openings are formed in the insulating films, hydrogen is prevented from reaching either second electrode 106 or metal oxide layer 104 in first reference element 100c and second reference element 100d.

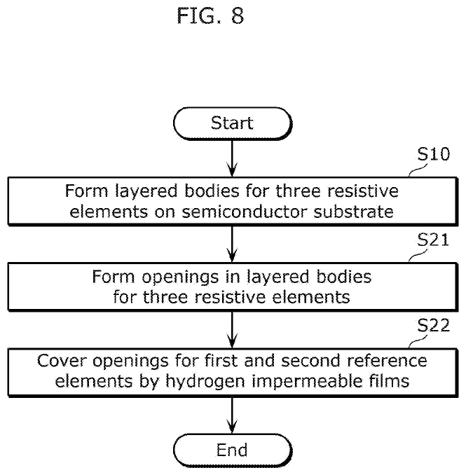

FIG. 8 is a flowchart illustrating a method for manufacturing sensor chip 10a illustrated in FIG. 7A and FIG. 7B. First, film formation and photolithography (pattern transferring and etching) for forming layered bodies for three resistive elements (hydrogen sensor element 100, first reference element 100c, and second reference element 100d) are repeatedly performed on semiconductor substrate 102 to form layered bodies for hydrogen sensor element 100, first reference element 100c, and second reference element 100d (layered body forming step S10). This step is the same as layered body forming step S10 shown in FIG. 3. In the present variation, by performing this step, openings 110, 110a, and 110b and the intermediate products of hydrogen sensor element 100, first reference element 100c, and second reference element 100d in which hydrogen-impermeable films 111a and 111b have not yet been formed are manufactured.

Next, photolithography (pattern transferring and etching) is performed on the layered bodies for the three resistive elements manufactured (i.e., the three intermediate products manufactured) to remove, in a rectangular shape, part of metal layer 106s, insulating film 107b, insulating film 109a, insulating film 107c, and insulating film 109b so as to expose at least part of the upper surface of second electrode 106, and thus opening 110 of hydrogen sensor element 100, opening 110a of first reference element 100c, and opening 110b of second reference element 100d are formed (opening forming step S21). By performing this step, hydrogen sensor element 100 is manufactured.

Finally, the inner side surfaces and the bottom surfaces of openings 110a and 110b formed in first reference element 100c and second reference element 100d are covered by hydrogen-impermeable films 111a and 111b each containing P-SiON or the like (hydrogen-impermeable film forming step S22). By performing this step, first reference element 100c and second reference element 100d provided with openings 110a and 110b including the inner side surfaces and the bottom surfaces covered by hydrogen-impermeable films 111a and 111b are manufactured. It should be noted that formation of hydrogen-impermeable films 111a and 111b may be performed in the same process as formation of insulating film 109b (i.e., film formation using the same material as insulating film 109b), or may be performed in an independent process. By performing this step, the manufacture of sensor chip 10a according to the variation of the embodiment is completed.

Even in hydrogen detection device 30 including, instead of sensor chip 10 according to the embodiment, sensor chip 10a manufactured as described above, signal processor 23 can generate, as a signal indicating hydrogen concentration, the voltage ratio represented by Expression 1, that is, Expression 2, in accordance with the flowchart illustrated in FIG. 4, in the same manner as the embodiment. In other words, even in the present variation, temperature dependence is suppressed, the component of unnecessary external resistance Rext in hydrogen sensor element 100 is removed, and a signal indicating hydrogen concentration with high sensitivity can be generated, in the same manner as the embodiment.

It should be noted that although hydrogen detection device 30 is a measurement system that includes sensor chip 10 or 10a together with measurement unit 20 in the embodiment described above, hydrogen detection device 30 may be a device that includes only sensor chip 10 or 10a. This is because sensor chip 10 or 10a has a characteristic sensor structure that enables detecting hydrogen with high sensitivity in a state in which temperature dependence is suppressed.

As described above, hydrogen detection device 30 according to the present embodiment is a device that detects hydrogen and includes hydrogen sensor element 100, first reference element 100a, and second reference element 100b that are provided on semiconductor substrate 102 and connected in series. Each of hydrogen sensor element 100, first reference element 100a, and second reference element 100b includes: first electrode 103 including a principal surface and second electrode 106 including a principal surface, the principal surface of first electrode 103 and the principal surface of second electrode 106 facing each other; metal oxide layer 104 disposed in contact with the principal surface of first electrode 103 and the principal surface of second electrode 106; and insulating film 107c and the like covering first electrode 103, second electrode 106, and metal oxide layer 104, hydrogen sensor element 100 includes opening 110 that is not covered by insulating film 107c and the like and through which an other surface of second electrode 106 opposite to the principal surface is exposed to an external environment, each of first reference element 100a and second reference element 100b includes no opening through which an other surface of second electrode 106 opposite to the principal surface is exposed to the external environment, and first reference element 100a and second reference element 100b have different lengths in a direction in which a current flows.

Accordingly, since hydrogen detection device 30 includes a characteristic sensor chip that enables, by using the three characteristic resistive elements, detecting hydrogen with high sensitivity in a state in which temperature dependence is suppressed, it is possible to realize hydrogen detection device 30 that does not require four resistive elements as in a conventional one, is realized with a smaller area than that of a conventional one, and can detect hydrogen with high sensitivity. Accordingly, by configuring the reference resistive elements such that hydrogen does not reach either the second electrode or the metal oxide layer thereof, the reference resistive elements are prevented from undergoing catalytic hydrogen dissociation, thereby stably maintaining reference resistance characteristics.

Here, as described in the variation of the embodiment, first reference element 100c and second reference element 100d may include openings 110a and 110b each of which is not covered by insulating film 107c and the like and through each of which the other surface of second electrode 106 opposite to the principal surface is exposed from insulating film 107c and the like, and opening 110a of first reference element 100c and opening 110b of second reference element 100d may include inner side surfaces and bottom surfaces that are covered by hydrogen-impermeable films 111a and 111b. Accordingly, since the openings are provided in the three resistive elements, structural identity is ensured at an extremely high level, and the effect of suppressing temperature dependence can be enhanced.

Moreover, hydrogen sensor element 100 and first reference element 100a may have the same length in the direction in which the current flows. Accordingly, a voltage ratio using the two resistive elements, that is, a resistance ratio, becomes approximately 1 when there is no hydrogen, and the accuracy of a signal indicating hydrogen concentration is ensured.

Moreover, one of first reference element 100a or second reference element 100b may be at least twice as long as that of the other in the direction in which the current flows. Accordingly, it is possible to ensure the calculation accuracy of calculating voltage Vext due to external resistance Rext based on the linear relationship between the lengths of the internal resistances of the two reference elements and the voltages each of which is a voltage across a different one of the two reference elements, and as a result, it is possible to calculate a signal indicating hydrogen concentration with high accuracy by using hydrogen detection device 30.

Moreover, hydrogen detection device 30 may further include: current source 21 that applies the current to hydrogen sensor element 100, first reference element 100a, and second reference element 100b that are connected in series; voltmeter 22 that measures a voltage across both ends of each of hydrogen sensor element 100, first reference element 100a, and second reference element 100b when the current is applied by current source 21; and signal processor 23 that generates a signal indicating hydrogen concentration by using the voltage across both ends of each of hydrogen sensor element 100, first reference element 100a, and second reference element 100b obtained by voltmeter 22, and outputs the signal. Accordingly, by taking advantages of the characteristic of sensor chip 10 including the three resistive elements, a signal indicating hydrogen concentration with high sensitivity is generated in a state in which temperature dependence is suppressed.

Here, a resistance of hydrogen sensor element 100 includes: resistance component Rs at a portion located at opening 110 in hydrogen sensor element 100; and external resistance Rext that is a resistance component excluding the resistance component at the portion, and signal processor 23 calculates voltage Vext generated by external resistance Rext by using voltage Vf1 across both ends of first reference element 100a and voltage Vf2 across both ends of second reference element 100b, and generates the signal indicating the hydrogen concentration by using voltage Vext calculated. Accordingly, an unnecessary voltage due to external resistance Rext is excluded and a signal indicating hydrogen concentration with high sensitivity is generated.

Specifically, signal processor 23: calculates a ratio represented by (Vh−Vext)/(Vf−Vext) where Vext denotes the voltage generated by the external resistance, Vh denotes the voltage across both ends of hydrogen sensor element 100, and Vf denotes the voltage across both ends of first reference element 100a; and generates, as the signal indicating the hydrogen concentration, a signal indicating the ratio calculated. Accordingly, by using a simple expression, an unnecessary voltage due to external resistance Rext is excluded and a signal indicating hydrogen concentration with high sensitivity is generated.

Moreover, a hydrogen detection method according to the present embodiment is a method for detecting hydrogen and includes: preparing the above-described hydrogen detection device 30; current applying step S30 of applying a current to hydrogen sensor element 100, first reference element 100a, and second reference element 100b that are included in hydrogen detection device 30 prepared and are connected in series; voltage measuring step S31 of measuring a voltage across both ends of each of hydrogen sensor element 100, first reference element 100a, and second reference element 100b when the current is applied in current applying step S30; and signal processing step S32 of generating a signal indicating hydrogen concentration by using the voltage across both ends of each of hydrogen sensor element 100, first reference element 100a, and second reference element 100b obtained in voltage measuring step S31, and outputting the signal.

Accordingly, by using a sensor chip that includes the three characteristic resistive elements and is implemented in a smaller area than a conventional one, a signal indicating hydrogen concentration with high sensitivity is generated.

Hereinabove, although the hydrogen detection device and the hydrogen detection method according to the present disclosure have been described based on the embodiment and variation, the present disclosure is not limited to these embodiment and variation. Various modifications of the embodiment and variation as well as other embodiments resulting from combinations of some of the constituent elements in the embodiment and variation that may be conceived by those skilled in the art are included within the scope of the present disclosure as long as they do not depart from the essence of the present disclosure.

For example, although a signal indicating hydrogen concentration is calculated based on Expression 1 described above using voltage Vh across both ends of hydrogen sensor element 100 and voltage Vf1 across both ends of first reference element 100a in the embodiment or the like, the present disclosure is not limited to this expression, and, instead of this expression, as shown in Expression 3 below, a signal indicating hydrogen concentration may be calculated by using voltage Vh across both ends of hydrogen sensor element 100 and voltage Vf2 across both ends of second reference element 100b.

(Vh−Vext)/(Vf2−Vext) (expression 3)

Moreover, although voltage Vext generated by external resistance Rext is calculated based on a straight line passing through two points using two reference elements (first reference element 100a and second reference element 100b or the like) in the embodiment or the like, the present disclosure is not limited to the two points, and voltage Vext generated by external resistance Rext may be calculated based on an approximate straight line passing through three or more points using three or more reference elements that are connected to hydrogen sensor element 100 in series in sensor chip 10 and have mutually different lengths.

Furthermore, the present disclosure may be realized as a program for causing a processor to execute the steps included in the hydrogen detection method according to the embodiment, as a non-transitory recording medium, such as a DVD, that has recorded thereon such a program, or as a program product that includes such a program.

Although only some exemplary embodiments of the present disclosure have been described in detail above, those skilled in the art will readily appreciate that many modifications are possible in the exemplary embodiments without materially departing from the novel teachings and advantages of the present disclosure. Accordingly, all such modifications are intended to be included within the scope of the present disclosure.

### INDUSTRIAL APPLICABILITY

The present disclosure can be utilized as a hydrogen detection device that includes a sensor chip containing resistive elements provided on a semiconductor substrate, for example, as a hydrogen detection device that is provided to a hydrogen vehicle or a hydrogen station and detects hydrogen leakage.

1. A hydrogen detection device configured to detect hydrogen, comprising:
a first resistive element, a second resistive element, and a third resistive element provided on a semiconductor substrate and connected electrically in series,
wherein each of the first resistive element, the second resistive element, and the third resistive element includes:
a first electrode including a principal surface and a second electrode including a principal surface, the principal surface of the first electrode and the principal surface of the second electrode facing each other;
a metal oxide layer disposed in contact with the principal surface of the first electrode and the principal surface of the second electrode; and
an insulating film covering the first electrode, the second electrode, and the metal oxide layer,
wherein the first resistive element includes an opening that is not covered by the insulating film and through which an other surface of the second electrode opposite to the principal surface is exposed to an external environment,
wherein each of the second resistive element and the third resistive element includes no opening through which an other surface of the second electrode opposite to the principal surface is exposed to the external environment, and
the second resistive element and the third resistive element have different lengths in a direction in which a current flows.

2. The hydrogen detection device according to claim 1,
wherein each of the second resistive element and the third resistive element includes an opening that is not covered by the insulating film and through which the other surface of the second electrode opposite to the principal surface is exposed from the insulating film, and
wherein the opening of each of the second resistive element and the third resistive element includes an inner side surface and a bottom surface covered by a hydrogen-impermeable film.

3. The hydrogen detection device according to claim 1, wherein
the first resistive element and the second resistive element have a same length in the direction in which the current flows.

4. The hydrogen detection device according to claim 1, wherein
one of the second resistive element or the third resistive element has a length at least twice as long as that of the other in the direction in which the current flows.

5. The hydrogen detection device according to claim 1, further comprising:
a current source that applies the current to the first resistive element, the second resistive element, and the third resistive element that are connected in series;
a voltmeter that measures a voltage across both ends of each of the first resistive element, the second resistive element, and the third resistive element when the current is applied by the current source; and
a signal processor that generates a signal indicating hydrogen concentration by using the voltage across both ends of each of the first resistive element, the second resistive element, and the third resistive element obtained by the voltmeter, and outputs the signal.

6. The hydrogen detection device according to claim 5, wherein
a resistance of the first resistive element includes: a resistance component at a portion located at the opening in the first resistive element; and an external resistance that is a resistance component excluding the resistance component at the portion, and
the signal processor calculates a voltage generated by the external resistance by using the voltage across both ends of each of the second resistive element and the third resistive element, and generates the signal indicating the hydrogen concentration by using the voltage calculated.

7. The hydrogen detection device according to claim 6, wherein
the signal processor: calculates a ratio represented by (Vh−Vext)/(Vf−Vext) where Vext denotes the voltage generated by the external resistance, Vh denotes the voltage across both ends of the first resistive element, and Vf denotes the voltage across both ends of the second resistive element; and generates, as the signal indicating the hydrogen concentration, a signal indicating the ratio calculated.

8. A hydrogen detection method comprising:
preparing the hydrogen detection device according to claim 1;
applying a current to the first resistive element, the second resistive element, and the third resistive element that are included in the hydrogen detection device prepared and are connected in series;
measuring a voltage across both ends of each of the first resistive element, the second resistive element, and the third resistive element when the current is applied in the applying; and
generating a signal indicating hydrogen concentration by using the voltage across both ends of each of the first resistive element, the second resistive element, and the third resistive element obtained in the measuring, and outputting the signal.
